(19) United States
(12) Patent Application Publication
(10) Pub. No.: US 2025/0264384 A1
(43) Pub. Date: Aug. 21, 2025

(54) SPECIMEN POLISHING APPARATUS

(71) Applicant: SAMSUNG ELECTRONICS CO., LTD., Suwon-si (KR)

(72) Inventors: Seungchul HAN, Suwon-si (KR); Wongyu JUNG, Suwon-si (KR); Sungha WOO, Suwon-si (KR); Jungjun PARK, Suwon-si (KR); Hoseok SONG, Suwon-si (KR); Sungmin CHO, Suwon-si (KR)

(21) Appl. No.: 18/805,251

(22) Filed: Aug. 14, 2024

(30) Foreign Application Priority Data
Feb. 15, 2024 (KR) 10-2024-0022014

(51) Int. Cl. G01N 1/32; G01N 33/00

(52) U.S. Cl. CPC G01N 1/32; G01N 33/0095

(57) ABSTRACT

A specimen polishing apparatus configured to polish a specimen in contact with an upper surface of a platen includes a hanger in adjacent paced-apart relationship with an upper portion of the platen; a holder coupled to the hanger and configured to support the specimen so that an observation surface of the specimen faces the upper surface of the platen; and a driving portion coupled to the hanger and configured to move the hanger along a z-axis direction that is perpendicular to the upper surface of the platen. An orientation of the holder relative to the platen upper surface is adjusted through rotation around a y-axis direction that is parallel to a longitudinal direction of the observation surface of the specimen and rotation around an x-axis direction that is perpendicular to the z-axis and the y-axis.

### CROSS-REFERENCE TO RELATED APPLICATION

This application claims priority to and the benefit of Korean Patent Application No. 10-2024-0022014 filed in the Korean Intellectual Property Office on Feb. 15, 2024, the disclosure of which is incorporated herein by reference in its entirety.

### BACKGROUND OF THE INVENTION

### (a) Field of the Invention

The present disclosure relates to a specimen polishing apparatus.

### (b) Description of the Related Art

Recently, as semiconductor technology has become more advanced and the types of products have become more diverse, inspections to check the profile of specimen patterns are becoming important.

A scanning electron microscope (SEM) is equipment that analyzes the structure and surface of a specimen by scanning the surface of the specimen and generating a sample image. The SEM has the advantage of being able to observe at high magnification because it has high resolution.

To analyze the cross-section of the specimen using vertical scanning electron microscopy (VSEM), a specimen for analysis must be produced, and the specimen for analysis is generally manufactured by cutting it into a quadrangular shape using a laser. However, since dust and a heat affected zone (HAZ) exist on the cut surface of the specimen manufactured in this way, it is difficult to measure the critical dimension (CD) of the specimen pattern to be observed using the VSEM.

Another method of manufacturing a specimen for analysis is to polish the cross-section of the specimen to be observed. However, the shape of the manufactured specimen itself may be distorted during the polishing process, or errors such as slope may occur during the process of mounting the specimen to the polishing apparatus, resulting in problems such as polishing verticality, horizontality, and cracking of the specimen.

Regardless of the shape of the specimen itself and the error that occurs in the process of mounting the specimen to the specimen polishing apparatus, there is a need for a technology that allows the observation surface of the specimen to be uniformly polished.

### SUMMARY OF THE INVENTION

Embodiments of the present disclosure provide a holder that supports a specimen and a hanger that supports the holder, wherein the holder has a degree of freedom to rotate in a longitudinal direction (y-axis) along which an observation surface of the specimen extends and in a vertical direction (x-axis) on the same plane as the longitudinal direction, a specimen polishing apparatus that may uniformly polish the observation surface of the specimen regardless of a shape of the specimen and any errors that occur during the process of mounting the specimen to the specimen polishing apparatus.

An embodiment provides a specimen polishing apparatus configured to polish a specimen that is in contact with an upper surface of a platen. The specimen polishing apparatus includes a hanger in adjacent spaced-apart relationship with the upper surface of the platen; a holder coupled to the hanger, wherein the holder is configured to support the specimen so that an observation surface of the specimen faces the upper surface of the platen; and a driving portion coupled to the hanger, wherein the driving portion is configured to move the hanger along a z-axis direction that is perpendicular to the upper surface of the platen, wherein an orientation of the holder is adjusted through rotation of the holder around a y-axis direction that is parallel to a longitudinal direction of the observation surface of the specimen and around an x-axis direction that is perpendicular to the z-axis and the y-axis.

Another embodiment provides a specimen polishing apparatus that includes a platen; a hanger in adjacent spaced-apart relationship with an upper surface of the platen; a holder coupled to the hanger, wherein the holder is configured to support a specimen so that an observation surface of the specimen faces the upper surface of the platen; a driving portion coupled to one side of the hanger, wherein the driving portion is configured to move the hanger along a z-axis direction that is perpendicular to the upper surface of the platen; and a rotation driving portion below the platen, wherein the driving portion is configured to rotate the platen around the z-axis, wherein the holder is rotatable around a y-axis direction that is parallel to a longitudinal direction of the observation surface of the specimen and is rotatable around an x-axis direction that is perpendicular to the z-axis and the y-axis.

Another embodiment provides a specimen polishing apparatus that is configured to polish a specimen that is in contact with an upper surface of a platen. The specimen polishing apparatus includes a hanger that supports a lower surface of a holder facing the platen, wherein the hanger includes a first support portion with a protrusion that extends toward the lower surface of the holder, a second support portion having one side that includes a rotation coupling portion and an opposite side that is connected to one side of the first support portion, and a third support portion having one side coupled to the driving portion and an opposite side having a cavity in which the rotation coupling portion is rotationally coupled; a holder configured to support the specimen so that an observation surface of the specimen faces an upper surface of the platen, wherein a longitudinal direction of the observation surface is parallel to a y-axis direction, wherein the lower surface of the holder includes a groove having a length that extends along the y-axis direction, and wherein an end of the protrusion is in contact with the groove; and a driving portion coupled to one side of the hanger, wherein the driving portion is configured to move the hanger along a z-axis direction that is perpendicular to the upper surface of the platen.

According to the embodiments, because the holder supporting the specimen has a degree of freedom in two directions, the pressure applied between the observation surface of the specimen in contact with the platen and the platen may be equally adjusted, and even if the disposition structure of the specimen supported by the holder is different each time, all surfaces of the observation surface of the specimen may be uniformly polished to minimize uneven wear.

### BRIEF DESCRIPTION OF THE DRAWINGS

FIG. 1 illustrates a specimen polishing apparatus according to an embodiment.

FIG. 2 illustrates a cross-section of a portion of the specimen polishing apparatus according to FIG. 1.

FIG. 3 illustrates a holder of a specimen polishing apparatus according to an embodiment.

FIG. 4 illustrates a holder of a specimen polishing apparatus according to an embodiment.

FIG. 5 illustrates a hanger of a specimen polishing apparatus according to an embodiment.

FIG. 6 illustrates the coupling of a holder and a hanger in a specimen polishing apparatus according to an embodiment.

FIG. 7 illustrates the coupling of a holder and a hanger in a specimen polishing apparatus according to an embodiment.

FIG. 8 illustrates a specimen polishing apparatus according to an embodiment.

FIG. 9 illustrates a specimen polishing apparatus according to an embodiment.

FIG. 10 illustrates a specimen polishing apparatus according to another embodiment.

### DETAILED DESCRIPTION OF THE EMBODIMENTS

The present disclosure will be described more fully hereinafter with reference to the accompanying drawings, in which embodiments of the present disclosure are shown. As those skilled in the art would realize, the described embodiments may be modified in various different ways, all without departing from the scope of the present disclosure.

In order to clearly describe the present disclosure, parts that are irrelevant to the description are omitted, and identical or similar constituent elements throughout the specification are denoted by the same reference numerals.

Further, in the drawings, the size and thickness of each element are arbitrarily illustrated for ease of description, and the present disclosure is not necessarily limited to those illustrated in the drawings. In the drawings, the thicknesses of layers, films, panels, regions, areas, etc., may be exaggerated for clarity. In the drawings, for ease of description, the thicknesses of some layers and areas may be exaggerated.

Throughout this specification and the claims that follow, when it is described that an element is “coupled or connected” to another element, the element may be “directly coupled or connected” to the other element or “indirectly coupled or connected” to the other element through a third element. In addition, unless explicitly described to the contrary, the word “comprise” and variations such as “comprises” or “comprising” will be understood to imply the inclusion of stated elements but not the exclusion of any other elements.

It will be understood that when an element such as a layer, film, region, area, substrate, plate, or the like is referred to as being “on” another element, it can be directly on the other element or intervening elements may also be present. In contrast, when an element is referred to as being “directly on” another element, there are no intervening elements present. Further, in the specification, the word “on” or “above” means disposed on or below the object portion, and does not necessarily mean disposed on the upper side of the object portion based on a gravitational direction.

Further, throughout the specification, the phrase “in plan view” or “on a plane” means viewing a target portion from above, and the phrase “in a cross-sectional view” or “on a cross-section” means viewing a cross-section formed by vertically cutting a target portion from a side.

In a conventional method of polishing a cross-section of a specimen to be observed, a method of fixing the specimen includes molding the specimen and then placing the molded specimen on a polishing apparatus. However, since a current must flow through the specimen for SEM imaging, the SEM imaging is substantially impossible by the method of fixing the specimen through the molding as described above.

For SEM imaging, if a specimen is polished after fixing the specimen using a specimen fixing device that fixes the specimen, it is essential to align the fixing device that fixes the specimen so that the desired observation surface may be obtained in the process of polishing the specimen according to the mounting shape of the specimen.

However, it is difficult to determine in which direction and how much the fixing device that fixes the specimen should be adjusted, and the precision of the degree of adjustment is lowered. In addition, errors inevitably occur in the alignment process, and repeating the corresponding task every time may reduce the efficiency of the task.

A specimen polishing apparatus 10 according to the present disclosure is intended to improve the problem of the prior art, and aims to uniformly polish the observation surface of a specimen regardless of the shape of the specimen itself and any errors that occur in the process of mounting the specimen to the specimen polishing apparatus. For this purpose, particularly, the device that supports the specimen is automatically aligned, so the alignment operation does not need to be repeatedly performed, thereby improving the efficiency of the work.

Hereinafter, a specimen polishing apparatus 10 according to an embodiment of the present disclosure will be described in more detail with reference to the accompanying drawings.

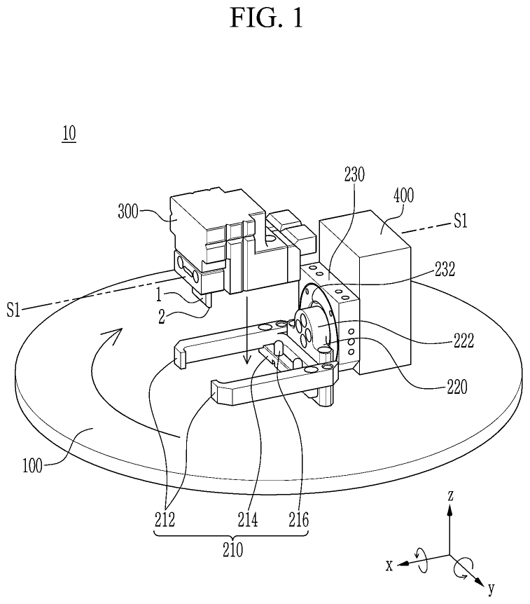

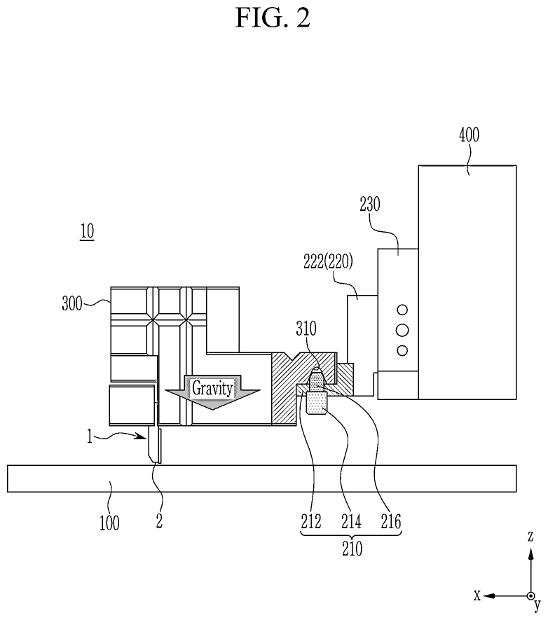

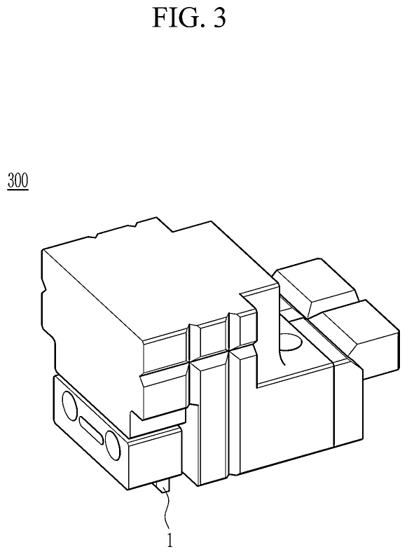

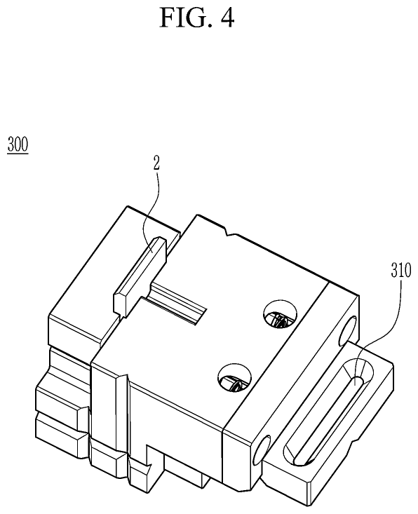

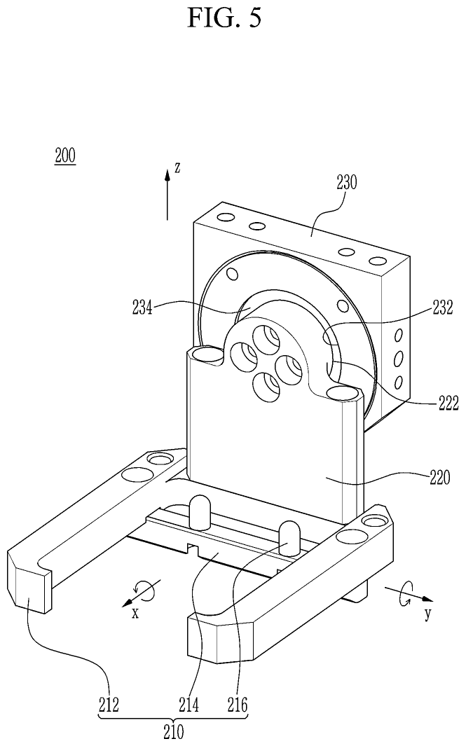

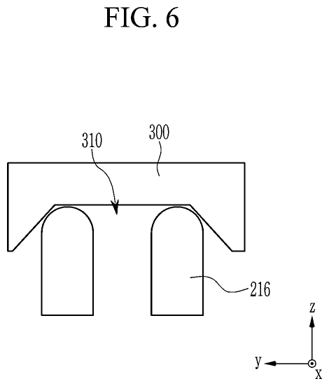

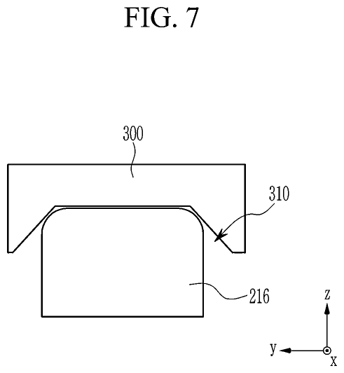

FIG. 1 illustrates a specimen polishing apparatus according to an embodiment, FIG. 2 illustrates a cross-section taken along line S1-S1 in FIG. 1, FIG. 3 and FIG. 4 illustrate a holder of a specimen polishing apparatus according to an embodiment, FIG. 5 illustrates a hanger of a specimen polishing apparatus according to an embodiment, and FIG. 6 and FIG. 7 illustrate coupling of a holder and a hanger in a specimen polishing apparatus according to an embodiment.

Referring to FIG. 1 to FIG. 5, the specimen polishing apparatus 10 according to the present disclosure may polish a specimen 1 in contact with an upper surface of a platen 100, and may include a hanger 200, a holder 300 coupled to the hanger 200, and a driving portion 400 coupled to the hanger 200 to move the hanger 200 along a z-axis direction that is perpendicular to the upper surface of the platen 100. The driving portion 400 is configured to move the hanger 200 relative to the platen 100 along the z-axis direction (i.e., toward and away from the platen) and may be a motor-driven device, such as a linear actuator, etc.

The hanger 200 is disposed on the upper portion of the platen 100 (e.g., in adjacent spaced apart relationship with the upper surface of the platen 100) and serves to support a lower surface of the holder 300 facing the platen 100.

FIG. 3 and FIG. 4 illustrate the upper and lower portions of the holder. The holder 300 serves to mount or support the specimen 1 so that an observation surface 2 of the specimen 1 faces the upper surface of the platen 100.

The holder 300 adjusts a slope of the holder 300 (i.e., adjusts an orientation of the holder 300 relative to the upper surface of the platen 100) through rotation around a y-axis direction parallel to the longitudinal direction along which the observation surface 2 of the specimen 1 extends and an x-axis direction perpendicular to the z-axis and y-axis.

The specimen polishing apparatus 10 according to the present disclosure moves vertically along the z-axis direction and may rotate around the x-axis and around the y-axis perpendicular to the z-axis.

Generally, rotation around the x-axis is called roll, rotation around the y-axis is called pitch, and in the present disclosure, the holder 300 has a degree of freedom of the roll and the pitch. The specimen 1 mounted on the holder 300 also has the same degree of freedom as a result of being supported by the holder 300.

The rotation around the x-axis is called the roll, and the rotation around the y-axis is called the pitch, but in practice, the holder 300 in the present disclosure may rotate by a certain angle of shaking in both directions with respect to respective axes.

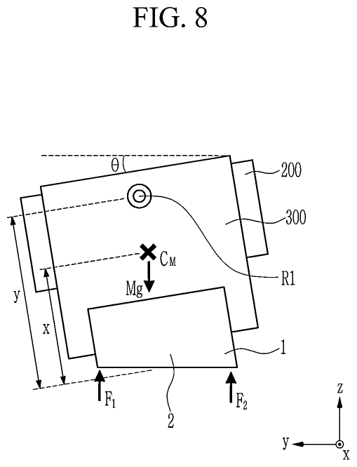

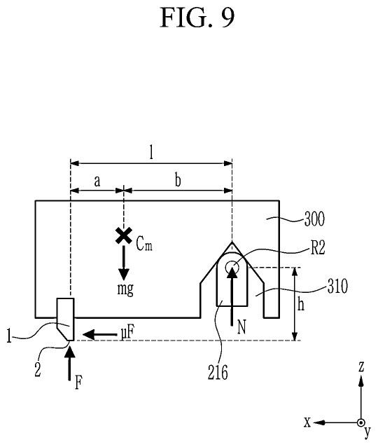

Although it will be described in detail with reference to FIG. 8 and FIG. 9, the specimen polishing apparatus 10 according to the present disclosure does not need to manually adjust and align the apparatus to obtain the desired observation surface 2.

This is because the holder 300 with the degree of freedom of the roll and the pitch automatically maintains the slope (i.e., the orientation) of the specimen 1 supported by the holder 300 in parallel with the platen 100, and the specimen 1 is polished by the same thickness on all observation surfaces 2 in contact with the platen 100.

The hanger 200 of the specimen polishing apparatus 10 according to the present disclosure may include a first support portion 210 supporting the lower surface of the holder 300 facing the platen 100, a second support portion 220 connected to one side of the first support portion 210, and a third support portion 230 coupled to one side of the second support portion 220.

As illustrated in FIG. 1, FIG. 2, and FIG. 5, the first support portion 210 may be disposed to have a length parallel to the x-axis direction, and may include a pair of first base portions 212 having one side connected to the second support portion 220, and a second base portion 214 that is disposed to have a length parallel to the y-axis direction and has both ends respectively connected to the pair of first base portions 212. A protrusion 216 that is disposed on the upper surface of the second base portion 214 and has a protruding shape may be included.

A rotation coupling portion 222 may be provided at one side of the second support portion 220, and the rotation coupling portion 222 may be coupled to the third support portion 230. In some embodiments, the rotation coupling portion 222 may have a length in the x-axis direction.

The third support portion 230 may include a hole or cavity 232 into which the rotation coupling portion 222 is inserted, and the rotation coupling portion 222 may have a structure in which the rotation coupling portion 222 is inserted into the cavity 232 as shown in FIG. 1.

As the rotation coupling portion 222 is inserted into the cavity 232, the second support portion 220 and the third support portion 230 may be coupled, and in some embodiments, a bearing 234 may be disposed between the rotation coupling portion 222 of the second support portion 220 and the cavity 232 of the third support portion 230. The illustrated cavity 232 is a cylindrical bore and the bearing 234 is secured (e.g., press fit, or otherwise secured, etc.) within the cylindrical bore. The rotation coupling portion 222 has a cylindrical shape that corresponds with the internal bore of the bearing 234 and is secured (e.g., press fit or removably secured, etc.) within the internal bore of the bearing 234. The coupling of the rotating coupling portion 222 to the bearing 234 allows the rotation coupling portion 222 to rotate relative to the third support portion 230.

The second support portion 220 may be rotated based on the x-axis (i.e., the second support portion 220 may rotate about the x-axis). In some embodiments, it may be rotated based on the x-axis, which is the longitudinal direction in which the rotation coupling portion 222 is disposed.

In this case, the first support portion 210 connected to the second support portion 220 and the holder 300 coupled to the upper end of the first support portion 210 may also rotate along with the second support portion 220 based on the x-axis (i.e., about the x-axis), which is the direction in which the rotation coupling portion 222 is disposed.

That is, the first support portion 210, the second support portion 220, and the holder 300 rotate together according to the rotation (Roll) based on the x-axis of the rotation coupling portion 222. Due to the rotation (Roll) around the x-axis, the observation surface 2 in contact with the platen 100 of the specimen 1 mounted on the holder 300 may be automatically aligned so that the same pressure and force are applied in all portions.

For example, assuming that the observation surface 2 of the specimen 1 and the platen 100 are not disposed parallel to each other, at least some of the observation surface 2 will come into contact with platen (100) first.

In this case, the holder 300 rotates through the rotation (Roll) around the x-axis described above, and through this rotation, instead of only a portion of the observation surface 2 being in contact with the platen 100, all surfaces of the observation surface 2 that finally comes into contact with the platen 100 may be made to be in equal contact with the platen 100.

Accordingly, it is automatically aligned so that the same pressure is applied to all portions of the observation surface 2 in contact with the platen 100 of the mounted specimen 1, and all portions of the observation surface 2 in contact with the platen 100 may be polished to the same thickness.

Above, the degree of freedom of the roll, which is rotation around the x-axis, has been described, and hereinafter, the degree of freedom of the pitch, which is rotation around the y-axis, will be described.

The upper surface of the second base portion 214 of the first support portion 210 may include the protrusion 216 protruding from the upper surface of the second base portion 214. In this case, as shown in FIG. 2 and FIG. 4, a groove 310 into which the protrusion 216 is inserted may be disposed on the lower surface of the holder 300 disposed on the upper portion of the protrusion 216, and the end of the protrusion 216 in contact with the groove 310 may be spherical.

As the end of the protrusion 216 has a curved shape, the holder 300 may have a degree of freedom of the rotation around the y-axis direction, that is, of the pitch.

FIG. 6 and FIG. 7 are drawings for describing coupling of the holder 300 and the hanger 200, and illustrate an enlarged view of the holder 300 including the protrusion 216 disposed on the upper surface of the first support portion 210 and the groove 310 that is disposed on the upper portion of the protrusion 216 and into which the protrusion 216 is inserted. As illustrated, the groove 310 having a length along the y-axis direction may be disposed on the lower surface of the holder 300 in contact with the protrusion 216.

FIG. 6 illustrates two protrusions 216 disposed on the upper surface of the second base portion 214 as shown in FIG. 5, and when there are a plurality of protrusions 216, the protrusions 216 may include a plurality of protrusions, and the plurality of protrusions may be arranged on the second base portion 214 along the y-axis direction.

FIG. 7 illustrates a case in which the protrusion 216 has an elongate shape extending along the y-axis direction, which is the length direction of the second base portion 214, and the protrusion 216 has a shape of one protrusion 216.

As shown in FIG. 6 and FIG. 7, the groove 310 into which all of the protrusions 216 formed on the upper surface of the second base portion 214 may be inserted is disposed on the lower surface of the holder 300 in the y-axis direction (i.e., the groove 310 extends along the y-axis direction).

As described above, as the end of the protrusion 216 has a curved shape, the groove 310 is coupled to the protrusion 216, so that the holder 300 may have a degree of freedom in the pitch direction.

In other words, while the protrusion 216 is inserted into the groove 310, the holder 300 may rotate in the pitch direction based on the y-axis in which the protrusion 216 and the groove 310 are coupled.

The rotation based on the y-axis (i.e., about the y-axis), that is, an axis serving as the center of the pitch may be substantially disposed at the upper end of the protrusion 216.

In this way, the structure that rotates around the y-axis may serve to move the observation surface 2 of the specimen 1 mounted on one side of the holder 300 close to the platen 100.

Referring to FIG. 2, by inserting the protrusion 216 disposed on the first support portion 210 into the groove 310 formed on the lower surface of the holder 300, the holder 300 may be in a state of being disposed on the first support portion 210.

In this case, the position (right) where the groove 310 of the holder 300 is disposed is supported by the protrusion 216, but there is no separate support structure in the case of the position (left) where the specimen 1 is mounted on the holder 300, so that a force is applied in the direction toward the platen 100 by gravity in a downward direction according to the weight of the holder 300.

In this case, the specimen 1 mounted on the left side of the holder 300 serves to support the left side of the holder 300 to sit down, and in this process, the observation surface 2 of the specimen 1 may contact the upper surface of the platen 100 without a separate alignment work.

In summary, the holder 300 has a structure that rotates around the y-axis based on the protrusion 216 inserted into the groove 310, and particularly, the portion on which the specimen 1 is mounted in the holder 300 moves downward by gravity.

In this case, referring to FIG. 2, the holder 300 moves counterclockwise by a certain angle based on the y-axis until the moment when the observation surface 2 comes into contact with the platen 100. Accordingly, the observation surface 2 of the mounted specimen 1 comes into contact with the platen 100 without a separate alignment operation.

FIG. 8 and FIG. 9 are drawings for describing a specimen polishing apparatus according to an embodiment, and illustrate a state in which the specimen polishing apparatus 10 is disposed on the upper surface of the platen 100 while the holder 300 and the hanger 200 are coupled, so that all observation surfaces 2 of the specimen 1 are aligned to be in contact with the upper surface of the platen 100.

As the drawing in which the platen 100 disposed at the lower portion is omitted and some other components are simplified and illustrated, FIG. 8 illustrates a cross-sectional view in a direction perpendicular to S1 in FIG. 1, and FIG. 9 illustrates a cross-sectional view in a direction of S1 in FIG. 1.

In the present disclosure, while the protrusion 216 is inserted into the groove 310, the holder 300 may be rotated based on the y-axis (i.e., about the y-axis) where the protrusion 216 and the groove 310 are coupled (see FIG. 9), and at the same time, it may also be rotated based on the x-axis direction (i.e., about the y-axis) perpendicular to the y-axis (see FIG. 8). FIG. 8 and FIG. 9 illustrate that the observation surface 2 is disposed parallel to the platen 100 by substantially rotating the holder 300 in the roll and pitch directions.

As described above, the specimen polishing apparatus 10 according to the present disclosure is characterized in that the slope (i.e., the orientation) of the holder 300 is automatically adjusted through the rotation.

The holder 300 may be disposed so that the straight line distance (y) from the observation surface 2 (from the upper surface of the platen 100) to the center R1 of the rotation coupling 222 of the second support portion 220 is greater than the straight line distance (x) from the observation surface 2 (from the upper surface of the platen 100) to the center of gravity CM of the coupled holder 300 and hanger 200.

However, as illustrated in FIG. 8, when the observation surface 2 mounted on the holder 300 has an oblique shape so as not to be parallel to the lower surface of the holder 300, based on the position of the observation surface 2 disposed at the center between one end and the other end of the observation surface 2, the straight line distance (y) from the observation surface 2 to the center R1 of the rotation coupling portion 222 and the straight line distance (x) from the observation surface 2 to the center of gravity CM of the coupled holder 300 and the hanger 200 may be measured and compared.

When the lower surface of the holder 300 and the surface of the observation surface 2 are not parallel, the angle formed by the lower surface of the holder 300 and the observation surface 2 is referred to as θ. When the observation surface 2 is mounted on the holder 300 while having an inclined angle by θ with the lower surface of the holder 300, the holder 300 is automatically aligned in order to apply the same pressure to all observation surfaces 2 during the polishing process of the observation surface 2.

Accordingly, as shown in FIG. 8, the holder can be aligned to be inclined at the angle of θ. In this case, it may be seen that the holder 300 has been rotated by θ with respect to the center R1 of the rotation coupling portion 222, that is, with respect to the x-axis direction.

That is, as shown in FIG. 8, the center R1 of the rotation coupling portion 222 is disposed higher than the center of gravity CM of the coupled holder 300 and hanger 200, so that all surfaces of the observation surface 2 of the specimen 1 come into contact with the platen 100 uniformly, and at this time, respective pressures (forces) F1 and F2 applied to both ends of the observation surface 2 have the same pressure (force). Accordingly, it may be polished by the same thickness on all observation surfaces 2.

In addition, as shown in FIG. 9, in the disposition of the holder 300 and the hanger 200, the height (h) from the observation surface 2 (from the upper surface of the platen 100) to the center R2 of the protrusion 216 may be disposed to be closer (i.e., shorter) than the straight distance from the observation surface 2 (from the upper surface of the platen 100) to the center of gravity Cm of the holder 300.

In addition, the sum of the distance (a) from the center of gravity Cm of the holder 300 to the specimen 1 and the distance (b) from the center of gravity Cm of the holder 300 to the center R2 of the protrusion 216 may be disposed to be the distance from the center R2 of the protrusion 216 to the specimen 1.

This is to allow the holder 300 to be disposed in the middle of the hanger 200 just by disposing the holder 300 on the hanger 200 so that the protrusion 216 is inserted into the groove 310 of the holder 300. When disposed in the above structure, the holder 300 may be supported by the mounted specimen 1 because a slight rotational force (shaking) is generated in the holder 300 in the counterclockwise direction.

When the above disposition relationship is not achieved, a strong rotational force is generated that causes the holder 300 in FIG. 9 to completely rotate in the counterclockwise direction, which may be a problem.

When the mass of the holder 300 and the hanger 200 is referred to as M, the mass of the holder 300 is referred to as m, and a gravitational acceleration (g) occurs, in FIG. 8, vertical drag forces of F1 and F2 occur at both ends of the observation surface 2, and in FIG. 9, friction (μF) and vertical drag force (F=F1+F2) occur in the specimen 1, and vertical drag (N) also occurs in protrusion 216.

In the specimen polishing apparatus 10 according to the present disclosure, even if the slope of the observation surface 2 varies every time depending on the shape of the specimen 1 and the form in which the specimen 1 is mounted, as a condition for uniformly polishing all surfaces of the observation surface 2, the straight line distance (y) from the upper surface of the platen 100 to the center R1 of the rotation coupling portion 222 is calculated by Equation 1 below.

$$y = x \cdot M / ( M - \frac{mb}{l + h μ} ) \qquad [ Equation 1 ]$$

First, in FIG. 8, assuming that the holder 300 is inclined and force is applied only at both end points of the observation surface 2, when the forces F1 and F2 generated at both end points are the same, the entire observation surface 2 be uniformly polished.

Referring to FIG. 9, the force applied to the specimen 1 (down force=F) may be calculated by the relationship between the freedom of the pitch of the holder 300 and the weight (m) of the holder (300). Accordingly, after calculating F in FIG. 9, using the equations of F=F1+F2 and F1=F2 in FIG. 8, the result of Equation 1 may be calculated.

When the position of the center R1 of the rotation coupling portion 222, that is, the straight line distance from the center R1 of the rotation coupling portion 222 is disposed at y, the point calculated Equation 1, the observation surface 2 of the specimen 1 may be polished while maintaining the angle of θ that is initially inclined. Accordingly, there is an effect of significantly reducing the amount of uneven wear.

In addition, since the equation for calculating the straight line distance (y) from the platen 100 to the center R1 of the rotation coupling portion 222 does not include the θ variable, when the specimen 1 is polished using the specimen polishing apparatus 10 according to the present disclosure, all surfaces of the observation surface 2 may be polished uniformly even if the slope of the observation surface 2 varies each time depending on the shape of the specimen 1 and the form in which the specimen 1 is mounted.

In conclusion, the use of the specimen polishing apparatus 10 according to the present disclosure has the effect of minimizing the value of uneven wear compared to the conventional apparatus for polishing the specimen.

As an experimental example, four specimens each having a size of 7×7 mm were used as a set, and the results of uneven wear by the conventional polishing apparatus and the results of uneven wear according to the specimen polishing apparatus 10 according to the present disclosure were compared.

The uneven wear value was calculated to mean the slope value obtained by using straight line regression of the polishing amount, and the uneven wear value by the conventional polishing apparatus was −8.722 [μm/mm], and the uneven wear value by the specimen polishing apparatus according to the present disclosure was-0.02654 [μm/mm].

Through the above results, it can be confirmed that the specimen polishing apparatus 10 according to the present disclosure has a significantly reduced uneven wear value compared to the conventional apparatus. In addition, in the case of the specimen polishing apparatus 10 according to the present disclosure, the polishing amount is also 50 μm or more, which is expected to be useful for VSEM observation.

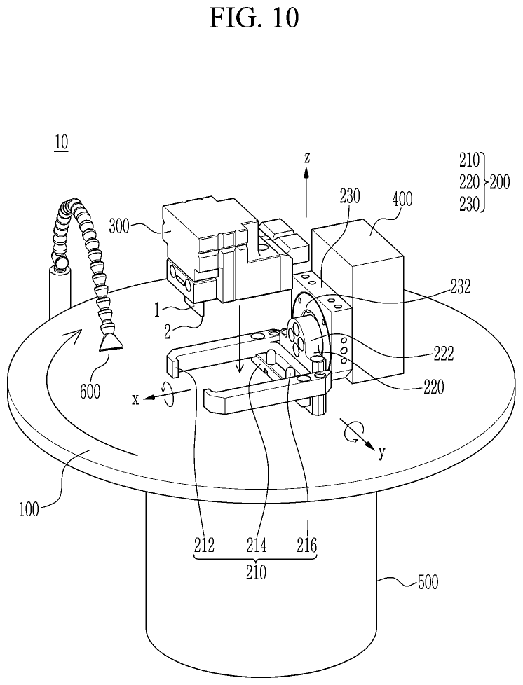

FIG. 10 illustrates a specimen polishing apparatus according to another embodiment.

As shown in FIG. 10, a specimen polishing apparatus 10 according to another embodiment may include a platen 100, a hanger 200 disposed on an upper portion of the platen 100, a holder 300 coupled to the hanger 200 and mounting a specimen 1 so that an observation surface 2 of the specimen 1 faces an upper surface of the platen 100, a driving portion 400 coupled to one side of the hanger 200 and moving the hanger 200 in the z-axis direction perpendicular to the platen 100, and a rotation driving portion 500 disposed below the platen 100 and rotating the platen 100 around the z-axis.

The holder 300 may rotate around y-axis direction parallel to the longitudinal direction in which the observation surface 2 of the specimen 1 is disposed, and around the x-axis direction perpendicular to the z-axis and the y-axis, and the slope of the holder 300 may be adjusted through the above rotation.

In other words, the specimen polishing apparatus 10 according to the present disclosure is characterized in that the holder 300 on which the specimen 1 is mounted is automatically aligned by the freedom of the roll, which is a rotation around the x-axis, and the freedom of the pitch, which is a rotation around the y-axis.

The specimen polishing apparatus 10 according to the present disclosure is characterized in that there is no need to align the device to obtain the desired observation surface 2, because the holder 300 with the degree of freedom of the roll and pitch may be polished by the same thickness on all observation surfaces 2 in contact with the platen 100 while maintaining the slope of the specimen 1 mounted on the holder 300 parallel to the platen 100.

The hanger 200 shown in FIG. 10 may include a first support portion 210 supporting the lower surface of the holder 300 facing the platen 100, a second support portion 220 in which one side includes a rotation coupling portion 222 and the other side is connected to one side of the first support portion 210, and a third support unit 230 in which one side is coupled to the driving portion 400 and a cavity 232 into which the rotation coupling portion 222 is inserted (and rotatably coupled with) is disposed on the other side.

In addition, the first support portion 210 may include a pair of first base portions 212 in which one side is connected to the second support portion 220 and that is disposed to have a length parallel to the x-axis direction, a second base portion 214 in which both ends thereof are respectively connected to the pair of first base portions 212 and that is disposed to have a length parallel to the y-axis direction, and a protrusion 216 protruding from the upper surface of the second base portion 214. A groove 310 may be disposed on the lower surface of the holder 300 in contact with the protrusion 216.

In the specimen polishing apparatus 10 shown in FIG. 10, the hanger 200 and the holder 300 are the same as those described in FIG. 1 to FIG. 9, so detailed descriptions thereof will be omitted.

According to the embodiment shown in FIG. 10, the specimen polishing apparatus 10 may further include an abrasive supply portion 600 that supplies abrasive to the upper surface of the platen 100.

The abrasive supply portion 600 serves to spray the abrasive toward the upper surface of the platen 100, and may allow the observation surface 2 to be polished by the abrasive during the process of polishing the observation surface 2 of the specimen 1.

In addition, although not shown, a supply portion for supplying cooling water and the like may be further included.

The specimen polishing apparatus 10 includes a rotation driving portion 500 (e.g., a motor or other device configured to impart rotational motion to the platen 100) disposed below the platen 100, and the platen 100, which is in contact with the observation surface 2 by the rotation driving portion 500, may be rotated around the z axis. The observation surface 2, which is disposed to be in contact with the upper surface of the platen 100, may be polished by rotation of the platen 100.

As described above, the specimen polishing apparatus 10 according to the present disclosure includes the holder 300 for mounting the specimen 1 and the hanger 200 for supporting the holder 300, and the holder 300 has a degree of freedom (pitch, roll) that allows rotation around the longitudinal direction (y-axis) in which the observation surface 2 of the specimen 1 is disposed and the direction (x-axis) perpendicular to the longitudinal direction, thereby uniformly polishing the observation surface of the specimen.

Particularly, in order to correct the shape of the specimen 1 itself and the error that occurs in the process of mounting the specimen 1 on the holder 300, the efficiency of the work may be increased in that the holder 300 is automatically aligned without a separate work of aligning the holder 300.

While this disclosure has been described in connection with what is presently considered to be practical embodiments, it is to be understood that the disclosure is not limited to the disclosed embodiments, but, on the contrary, is intended to cover various modifications and equivalent arrangements included within the scope of the appended claims.

What is claimed is:

1. A specimen polishing apparatus configured to polish a specimen that is in contact with an upper surface of a platen, the specimen polishing apparatus comprising:
a hanger in adjacent spaced-apart relationship with the upper surface of the platen;
a holder coupled to the hanger, wherein the holder is configured to support the specimen so that an observation surface of the specimen faces the upper surface of the platen; and
a driving portion coupled to the hanger, wherein the driving portion is configured to move the hanger along a z-axis direction that is perpendicular to the upper surface of the platen,
wherein an orientation of the holder relative to the upper surface of the platen is adjusted through rotation of the holder around a y-axis direction that is parallel to a longitudinal direction of the observation surface of the specimen and around an x-axis direction that is perpendicular to the z-axis and the y-axis.

2. The specimen polishing apparatus of claim 1, wherein
the hanger comprises:
a first support portion that supports a lower surface of the holder;
a second support portion connected to one side of the first support portion; and
a third support portion coupled to one side of the second support portion.

3. The specimen polishing apparatus of claim 2, wherein
the one side of the second support portion comprises a rotation coupling portion.

4. The specimen polishing apparatus of claim 3, wherein
the third support portion comprises a cavity into which the rotation coupling portion is rotatably coupled.

5. The specimen polishing apparatus of claim 4, wherein
the second support portion is rotatable around the x-axis.

6. The specimen polishing apparatus of claim 3, wherein
the first support portion and the holder coupled to the first support portion are rotatable together with the second support portion via the rotation coupling portion.

7. The specimen polishing apparatus of claim 4, wherein
a bearing is secured within the cavity between the rotation coupling portion of the second support portion and the cavity, and wherein the bearing facilitates rotation of the rotation coupling portion relative to the cavity.

8. The specimen polishing apparatus of claim 3, wherein
the first support portion comprises:
a pair of first base portions having respective lengths that are parallel to the x-axis direction, wherein a side of each of the pair of first base portions is connected to the second support portion;
a second base portion having a length that is parallel to the y-axis direction and in which respective ends thereof are connected to the pair of first base portions; and
at least one protrusion extending from an upper surface of the second base portion.

9. The specimen polishing apparatus of claim 8, wherein
the at least one protrusion has an elongate shape that extends along the y-axis direction.

10. The specimen polishing apparatus of claim 8, wherein
the at least one protrusion comprises a plurality of protrusions, wherein the plurality of protrusions are arranged along the y-axis direction on the second base portion.

11. The specimen polishing apparatus of claim 8, wherein
the lower surface of the holder comprises a groove having a length that extends along the y-axis direction, and wherein an end portion of the at least one protrusion is in contact with the groove, and
wherein the end portion of the at least one protrusion has a spherical shape.

12. A specimen polishing apparatus, comprising:
a platen;
a hanger in adjacent spaced-apart relationship with an upper surface of the platen;
a holder coupled to the hanger, wherein the holder is configured to support a specimen so that an observation surface of the specimen faces the upper surface of the platen;
a driving portion coupled to one side of the hanger, wherein the driving portion is configured to move the hanger along a z-axis direction that is perpendicular to the upper surface of the platen; and
a rotation driving portion below the platen, wherein the rotation driving portion is configured to rotate the platen around the z-axis,
wherein the holder is rotatable around a y-axis direction that is parallel to a longitudinal direction of the observation surface of the specimen and an x-axis direction that is perpendicular to the z-axis and the y-axis.

13. The specimen polishing apparatus of claim 12, further comprising
an abrasive supply portion that supplies abrasive to the upper surface of the platen.

14. The specimen polishing apparatus of claim 12, wherein
the hanger comprises:
a first support portion that supports a lower surface of the holder;
a second support portion, wherein one side of the second support portion comprises a rotation coupling portion and an opposite side of the second support portion is connected to one side of the first support portion; and
a third support portion, wherein one side of the third support portion is coupled to the driving portion and an opposite side of the third support portion comprises a cavity into which the rotation coupling portion is rotatably coupled.

15. The specimen polishing apparatus of claim 14, wherein
the first support portion comprises:
a pair of first base portions having respective lengths that are parallel to the x-axis direction, wherein a side of each of the pair of first base portions is connected to the second support portion;
a second base portion having a length that is parallel to the y-axis direction and, wherein respective ends of the second base portion are connected to the pair of first support portions; and
a protrusion extending from an upper surface of the second base portion.

16. The specimen polishing apparatus of claim 15, wherein
the lower surface of the holder comprises a groove, and wherein an end portion of the protrusion is in contact with the groove.

17. A specimen polishing apparatus configured to polish a specimen that is in contact with an upper surface of a platen, the specimen polishing apparatus comprising:
a hanger that supports a lower surface of a holder facing the platen, wherein the hanger comprises a first support portion with a protrusion that extends toward the lower surface of the holder, a second support portion having one side that comprises a rotation coupling portion and an opposite side that is connected to one side of the first support portion, and a third support portion having one side coupled to the driving portion and an opposite side having a cavity in which the rotation coupling portion is rotationally coupled;
a holder configured to support the specimen so that an observation surface of the specimen faces the upper surface of the platen, wherein a longitudinal direction of the observation surface is parallel to a y-axis direction, wherein the lower surface of the holder comprises a groove having a length that extends along the y-axis direction, and wherein an end of the protrusion is in contact with the groove; and
a driving portion coupled to one side of the hanger, wherein the driving portion is configured to move the hanger along a z-axis direction that is perpendicular to the upper surface of the platen.

18. The specimen polishing apparatus of claim 17, wherein
the holder is configured so that a straight line distance from a lower end of the holder to a center of the rotation coupling portion of the second support portion is longer than a straight line distance from the lower end of the holder to a center of gravity of the holder.

19. The specimen polishing apparatus of claim 17, wherein
the holder is configured so that a height from the lower end of the holder to a rotation axis of the protrusion is shorter than the straight line distance from the lower end of the holder to the center of gravity of the holder.

20. The specimen polishing apparatus of claim 17, wherein
the holder is rotatable around the y-axis and around the x-axis, and wherein an orientation of the holder relative to the upper surface of the platen is automatically adjusted by rotation of the holder about the x-axis and rotation of the holder about the y-axis.
